(19) United States
(12) Patent Application Publication
(10) Pub. No.: US 2012/0201912 A1
(43) Pub. Date: Aug. 9, 2012

(54) SUBSTANCES FOR REDUCING OCCURRENCE OF MAJOR CARDIAC EVENTS COMPRISING OMEGA-3 POLYUNSATURATED FATTY ACID OR DERIVATIVES THEREOF AND HIGH-MONACOLIN K CONTENT RED YEAST RICE EXTRACT

(76) Inventor: Joar A. Opheim, Aptos, CA (US)

(21) Appl. No.: 13/443,739

(22) Filed: Apr. 10, 2012

Related U.S. Application Data
(63) Continuation of application No. 13/420,410, filed on Mar. 14, 2012.
Application No. 13/420,410 is a continuation-in-part of application No. 12/630,686, filed on Dec. 3, 2009.
Application No. 12/630,686 is a continuation-in-part of application No. 11/757,340, filed on Jun. 1, 2007.

(51) Int. Cl. A61K 31/366; A61K 31/355; A61P 7/00; A61K 36/53

(52) U.S. Cl. 424/745; 514/460; 514/458

(57) ABSTRACT

Medicaments and therapeutic compositions comprise a composition (1) comprising at least one polyunsaturated fatty acid, at least one pharmaceutically acceptable derivative of a polyunsaturated fatty acid or mixtures thereof and a composition (2) comprising Red Yeast Rice extract comprising about 0.8 wt. % or more monacolin K. One source of component (1) is fish oil. The compositions are useful for lowering cholesterol and/or triglyceride levels in a subject.

### CROSS-REFERENCE TO RELATED APPLICATIONS

This application is a continuation of U.S. patent application Ser. No. 13/420,410, filed Mar. 14, 2012, which is a continuation-in-part of U.S. patent application Ser. No. 12/630,686, filed Dec. 3, 2009, which is a continuation-in-part of U.S. patent application Ser. No. 11/757,340, filed Jun. 1, 2007, both applications being incorporated herein by this reference in their entirety. All patents and patent applications cited in this application, all related applications referenced herein, and all references cited therein are incorporated herein by reference in their entirety as if restated here in full and as if each individual patent and patent application was specifically and individually indicated to be incorporated by reference.

### BACKGROUND OF THE INVENTION

1. Field of the Invention

The invention relates to compositions and the use of such compositions in therapeutic compositions, nutritional supplements and medicaments, wherein the compositions are combinations of (1) polyunsaturated fatty acids and/or derivatives thereof derived from fish oils or other sources of polyunsaturated fatty acids or derivatives thereof, and (2) Red Yeast Rice high in monacolin K content.

2. Description of Related Art

Statins (which are members of a group of HMG-CoA reductase inhibitors) are a group of hypolipidemic agents, used as pharmaceutical agents to lower cholesterol levels in people with or at risk for cardiovascular disease. Statins lower cholesterol by inhibiting the enzyme HMG-CoA reductase, which is the rate-limiting enzyme of the mevalonate pathway of cholesterol synthesis. Inhibition of this enzyme in the liver stimulates low-density lipoprotein (LDL) receptors, resulting in an increased clearance of LDL, so-called “bad cholesterol,” from the bloodstream and a decrease in blood cholesterol levels.

Statins are potent cholesterol-lowering agents, and have been reported to lower LDL cholesterol by 30-50% (see Jones et al., “Comparative dose efficacy study of atorvastatin versus simvastatin, pravastatin, lovastatin, and fluvastatin in patients with hypercholesterolemia (the CURVES study),” Am J Cardiol 1998; 81-(5), 582-7.) Statins are classified as either synthetic or fermentation derived. Lovastatin was isolated from a strain of Aspergillus terreus and it was the first statin approved by the FDA as a drug (August 1987). Lovastatin is a water insoluble, white crystalline solid. The aqueous insolubility of lovastatin leads to inadequate dissolution in gastrointestinal fluids and, hence, poor absorption, distribution, and targeted organ delivery. Solubility of lovastatin is enhanced by reaction with 3-cyclodextrin, an oligosaccharide which improves the solubility of lovastatin. The improvement of aqueous solubility in such a case is a valuable goal to improve therapeutic efficacy. Lovastatin can also produce slight to moderate increases in high density lipoproteins (HDL) (10-20%), and slight decreases in triglycerides (5-10%). The usual daily dose of lovastatin is 20-80 mg/day. The statin drugs include lovastatin, pravastatin, fluvastatin, atorvastatin, simvastatin, rosuvastatin, and cerivastatin.

Compounds similar to lovastatin have also been found in a natural fermentation product known as Red Yeast Rice. These compounds are also HMG-CoA reductase inhibitors. A monograph published in Alternative Medicine Review (Volume 9, Number 2, 2004) reports that the HMG-CoA reductase inhibitor activity in Red Yeast Rice comes from a naturally occurring family of nine compounds called “monacolins,” each of which has HMG-CoA reductase inhibitor activity. Additional active ingredients in Red Yeast Rice include sterols (beta-sitosterol, campesterol, sigmasterol, and sapogenin), isoflavones, and monounsaturated fatty acids (see Heber et al., “Cholesterol lowering effects of proprietary Chinese red yeast rice dietary supplement,” Am J Clin Nutr 1999, 69:231-236. One of the monacolins in Red Yeast Rice, monacolin K, is said to be the lactone form of the statin drug lovastatin, which is converted to the active acid form in vivo by the liver.

Red Yeast Rice is a common foodstuff in Asian countries where the average daily intake is 14-55 grams. The nutritional supplement derived from Red Yeast Rice is known as Red Yeast Rice extract. It is obtained by drying the fermented product of rice on which the yeast Monascus pupureus has been grown and extracting the dried product with a solvent, usually aqueous ethanol or water. The Red Yeast Rice extract thus produced typically contains about 0.2 wt. % monacolin K and about 0.5 wt. % total monacolins.

U.S. Pat. No. 6,046,022, issued Apr. 4, 2000, to Zhang et al., discloses some methods of making high lovastatin Red Yeast Rice and using Red Yeast Rice and Red Yeast Rice extract. U.S. Pat. No. 6,046,022 is hereby incorporated by reference herein in its entirety.

U.S. Pat. No. 6,541,005, issued Apr. 1, 2003, to Yegorova; U.S. Pat. No. 6,436,406, issued Aug. 20, 2002, to Yegorova; U.S. Pat. No. 6,495,173, issued Dec. 17, 2002, to Yegorova; U.S. Pat. No. 6,544,525, issued Apr. 8, 2003, to Yegorova; U.S. Pat. No. 6,576,242, issued Jun. 10, 2003, to Yegorova; U.S. Pat. No. 6,541,006, issued Apr. 1, 2003, to Yegorova; and U.S. Pat. No. 6,410,521, issued Jun. 25, 2002, to Mundy et al., disclose methods of using Red Yeast Rice. All of these patents are incorporated herein by reference in their entirety.

U.S. Patent Application Publication No. 2006/0211763, published Sep. 21, 2006, by Fawzy et al., discloses a statin drug dissolved in a solvent system comprising natural or synthetic omega-3 fatty acids, and U.S. Patent Application Publication No. 2006/0034815, published Feb. 16, 2006, by Guzman et al., discloses omega-3 oil solutions of one or more statins.

Omega-3 polyunsaturated fatty acids and derivatives thereof can be derived from fish oils and are known to reduce serum triglycerides (see Abe et al., “Soluble cell adhesion molecules in hypertriglyceridemia and potential significance on monocyte adhesion,” Arteriosler Thromb Vasc Biology 1998, 18:723-731) and adverse coronary events. The principal active ingredients in fish oil are 5,8,11,14,17-eicosapentaenoic acid (eicosapentaenoic acid (EPA), (20:5 (n-3)) and 4,7,10,13,16,19-docosahexaenoic acid (docosahexaenoic acid (DHA), 22:6 (n-3)). EPA and DHA have been given at a combined dose of 4 g/day for seven months to hypertriglyceridemic patients resulting in a reduction of 47% in triglycerides (see Ridker, Paul, “Effects of n-3 Fatty Acid Therapy on Lipids and sCAMs—Inflammatory Markers, Pharmacotherapy and Clinical Trials,” www.lipidsonline.org, posted Oct. 3, 2001, reviewed Oct. 4, 2001).

The effects of statin drugs and omega-3 polyunsaturated fatty acids or derivatives thereof have been reported to be cumulative. When 59 patients who were already receiving 10-40 mg daily of the statin simvastatin were given 2 grams twice daily of EPA+DHA, there was a further sustained significant decrease of 20-30% in triglycerides (see Durington et al., “An omega-3 polyunsaturated fatty acid concentration administered for one year decreased triglycerides in simvastin treated patients with CM,” Heart, 2001, 85(5) 544-548).

Fraser, 2001, Effect of Fish Oil and Red Yeast Rice Supplementation of Cardiovascular Disease Risk Factors in Hypercholesterolemic Men (Master's Thesis), retrieved from Dissertations and Theses Database (Publication ID MQ65930), discloses treatment of hypercholesterolemic men using fish oil and Red Yeast Rice, administered separately in separate capsules.

Omega-3 polyunsaturated fatty acids and derivatives thereof are also well known to those skilled in the art to reduce inflammation, decrease arrhythmias, decrease risk of sudden cardiac death and cardiac arrest.

### SUMMARY OF THE INVENTION

In accordance with the present invention there is provided a therapeutic composition comprising a composition (1) comprising at least one polyunsaturated fatty acid, at least one pharmaceutically acceptable derivative of a polyunsaturated fatty acid or mixtures thereof and a composition (2) comprising Red Yeast Rice extract comprising about 0.8 wt. % or more monacolin K. The Red Yeast Rice extract may comprise about 0.8 wt. % to about 25% monacolin K. In some embodiments, component (2) comprises 0.8 wt. % monacolin K, or about 2.4 wt. % monacolin K.

As used herein, the term “about” means that the value or amount to which it refers can vary by ±5%.

The present invention further provides therapeutic compositions wherein component (1) comprises at least one omega-3 polyunsaturated fatty acid, or at least one pharmaceutically acceptable omega-3 polyunsaturated fatty acid derivative or mixtures thereof. Component (1) can comprise EPA, pharmaceutically acceptable derivatives of EPA, DHA, pharmaceutically acceptable derivatives of DHA or mixtures thereof. The derivatives of EPA and/or DHA can be selected from the group consisting of esters, alkyl esters, glycerides and phospholipids. In some embodiments, the glycerides are triglycerides.

In some embodiments, the therapeutic compositions of the present invention include those wherein pharmaceutically acceptable derivatives of EPA and pharmaceutically acceptable derivatives of DHA are present in a weight ratio of about 3:1; about 3.2:1; about 3.3:1; about 5:1 or about 5.5:1. In some embodiments, the weight ratio is 3.3:1.

Also provided by the present invention are therapeutic compositions wherein component (1) is a mixture comprising about 35 wt. % triglycerides of EPA and about 25 wt. % triglycerides of DHA.

Further provided in accordance with the present invention are therapeutic compositions wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the EPA and DHA and the balance is at least about 80% mono- and di-glycerides. The component (1) combination can be at least about 80% in the triglyceride form, at least about 90% in the triglyceride form, at least about 98% in the triglyceride form, or at least about 98% in the triglyceride form and the remainder is monoglycerides, diglycerides or both.

Further provided in accordance with the present invention is a therapeutic composition wherein the weight ratio of component (1) to component (2) is in the range between about 1:1 to about 10:1 or between about 4:1 to about 10:1, wherein the weight ratio of component (1) to component (2) is about 4:1, and wherein the weight ratio of component (1) to component (2) is about 9:1.

The present invention also provides therapeutic compositions further comprising a dispersing agent, wherein the dispersing agent can comprise lysine and bamboo.

The therapeutic composition of the present invention can also include an antioxidant. The antioxidant can be chosen from the group consisting of rosemary, vitamin E, astaxanthine, carnitine, ascorbyl palmitate, and tocopherols.

The present invention also provides a daily dose of the therapeutic composition wherein the daily dose of therapeutic composition can comprise:
(i) about 2200 mg of component (1) and a sufficient amount of component (2) to provide at least about 6.0 mg of monacolin K, or
(ii) about 2700 mg of component (1) and a sufficient amount of component (2) to provide about 6.4 mg of monacolin K, or (iii) component (1) comprising about 2400 mg of a pharmaceutically
acceptable derivative of EPA and about 320 mg a pharmaceutically acceptable derivative of DHA; and a sufficient amount of component (2) to provide about 6.4 mg of monacolin K, or
(iv) component (1) comprising about 3240 mg of a pharmaceutically acceptable derivative of EPA and about 180 mg a pharmaceutically acceptable derivative of DHA; and a sufficient amount of component (2) to provide about 9.6 mg of monacolin K.

The present invention also includes soft gelatin capsule into which component (1), component (2) and dispersing agent are loaded, and wherein a daily dose of the therapeutic composition is delivered by an integral number of capsules.

In accordance with the present invention there is further provided a method of reducing serum cholesterol, triglycerides or both in a subject comprising administering to the subject an effective amount of a dosage comprising the above-recited therapeutic compositions.

### DETAILED DESCRIPTION OF EMBODIMENTS

The present invention relates to a therapeutic composition or medicament comprising a composition (1) comprising at least one polyunsaturated fatty acid, at least one pharmaceutically acceptable derivative of a polyunsaturated fatty acid or mixtures thereof (referred to herein as “component (1)”) and a composition (2) comprising Red Yeast Rice extract comprising about 0.8 wt. % or more monacolin K (referred to herein as “component (2)”). Component (2) can be dispersed in component (1) so that component (1) and component (2) are administered simultaneously, or component (1) and component (2) can be administered separately. Simultaneous administration is preferred.

In some embodiments, the therapeutic composition includes compositions in which component (1) comprises about 60% or more of omega-3 oils, or about 70% or more omega-3 oils in which the omega-3 oils contain component (1). In some embodiments, the therapeutic composition includes compositions which contain component (2) wherein component (2) comprises about 0.8 wt. % or more monacolin K. In some embodiments, the therapeutic composition includes compositions in which component (2) comprises about 2.4 wt. % or more monacolin K. In some embodiments, the therapeutic composition includes compositions in which component (2) comprises about 0.8 to about 25 wt. % monacolin K. These weight percentages are to be understood to include any weight percent between 0.8 wt % and 25 wt % in increments of 0.01 wt %. In some embodiments, the therapeutic composition has a weight ratio of component (1) to component (2) in the range between about 1:1 to about 10:1; in the range of about 4:1 to about 10:1; about 4:1 or about 9:1. These weight ratios are to be understood to include any weight ratio between 1:1 and 10:1 in increments of 0.01.

In some embodiments, the therapeutic composition further comprises a dispersing agent. In some embodiments, the dispersing agent comprises 3% lysine and 2% bamboo. In some embodiments, the therapeutic composition further comprises an antioxidant. In some embodiments, the antioxidant is chosen from the group consisting of rosemary, vitamin E, astaxanthine, carnitine, ascorbyl palmitate, and tocopherols. In some embodiments, the therapeutic composition further comprises a soft gelatin capsule into which the therapeutic composition and dispersing agent are loaded. In some embodiments, the therapeutic composition comprises a daily dose of the therapeutic composition which is delivered by an integral number of capsules.

In some embodiments, the daily dose of the therapeutic composition can comprise about 2200 mg of component (1) and a sufficient amount of component (2) to provide at least about 6.0 mg of monacolin K, or the daily dose can comprise about 2700 mg of component (1) and a sufficient amount of component (2) to provide about 6.4 mg of monacolin K. Also provided in accordance with the present invention are therapeutic compositions wherein a daily dose of the therapeutic composition comprises about 2400 mg of a pharmaceutically acceptable derivative EPA, about 320 mg of a pharmaceutically acceptable derivative of DHA, and a sufficient amount of component (2) to provide about 6.4 mg of monacolin K, or the daily dose can comprise about 3240 mg of a pharmaceutically acceptable derivative of EPA, about 180 mg of a pharmaceutically acceptable derivative of DHA, and a sufficient amount of component (2) to provide about 9.6 mg of monacolin K.

The present invention further relates to a method of reducing serum cholesterol, triglycerides or both in a subject comprising administering a daily dosage of a therapeutic composition of this invention. In some embodiments, the daily dosage comprises those recited above.

In some embodiments, the invention also relates to compositions comprising component (1) and component (2), and the use of such compositions to treat a subject. Component (2) comprises monacolin K. In some embodiments, component (2) comprises monacolin K and at least one other monacolin compound. In some embodiments, the monacolin compounds comprise all of the monacolin compounds in component (2).

The compositions of the present invention provide several advantages over the use of lovastatin to reduce cholesterol and triglyceride level in a subject. For instance, component (2) is water soluble, whereas lovastatin is not. As noted above, the water insolubility of lovastatin leads to inadequate dissolution in gastrointestinal fluids and, hence, poor absorption, distribution, and targeted organ delivery. While the water solubility of lovastatin can be enhanced, it is believed that the water soluble component (2) will enter the subjects system easier than lovastatin.

It is also emphasized that component (2) can produce better lipid reducing results at lower dosages (based on the amount of monacolin in component (2)) than lovastatin. This reduces the risk of undesirable and possibly harmful side effects in the subject.

Component (2) (the Red Yeast Rice extract) is prepared by fermenting white rice, preferably non-glutinous white rice, with Monascus purpureus strain of yeast by culturing said Monascus purpureus strain in a culture medium comprising rice at a temperature of about 15° C. to about 35° C. for a period of about 2 to about 20 days to provide a crude fermentation product containing Red Yeast Rice; drying said crude fermentation product to obtain Red Yeast Rice, extracting said Red Yeast Rice with a solvent to provide an extract; and drying said extract to remove the solvent and produce Red Yeast Rice extract. The solvent is preferably either aqueous ethanol or water. Other culture media may also be added to the rice. For example, sugar; an additional carbon source chosen from the group consisting of glycerine, malt, and potato juice; and thick beet juice or mixtures thereof may be used. In addition, a defoamer may be added.

In some embodiments, the Red Yeast Rice extract used in the therapeutic compositions of this invention contains relatively high levels of monacolin K. These high monacolin content Red Yeast Rice extracts can be prepared as described above with the fermentation step being continued to increase the monacolin K level to the desired amount.

In some embodiments, the Red Yeast Rice extract used in the compositions of the present invention contains about 0.8 wt. % or more monacolin K, about 0.8 to about 25 wt. % monacolin K, about 0.8 wt. % monacolin K, or about 2.4 wt. % monacolin K.

Red Yeast Rice extracts are readily available in commerce in the United States and may be purchased already prepared. Red Yeast Rice extracts containing higher amounts of monacolin K than are available commercially may be prepared as described above.

The polyunsaturated fatty acids can include omega-3 polyunsaturated fatty acids. As used herein, the term “omega-3 polyunsaturated fatty acid(s)” refers to a family of unsaturated fatty carboxylic acids that have in common a carbon-carbon bond in the n-3 position (i.e., the third bond from the methyl end of the molecule). Typically, they contain from about 16 to about 24 carbon atoms and from three to six carbon-carbon double bonds. Omega-3 polyunsaturated fatty acids can be found in nature, and these natural omega-3 polyunsaturated fatty acids frequently have all of their carbon-carbon double bonds in the cis-configuration.

Examples of omega-3 polyunsaturated fatty acids include, but are not limited to, 7,10,13-hexadecatrienoic acid (sometimes abbreviated as 16:3 (n-3)); 9,12,15-octadecatetrienoic acid (alpha-linolenic acid (ALA), 18:3 (n-3)); 6,9,12,15-octadecatetraenoic acid (stearidonic acid (STD), 18:4 (n-3)); 11,14,17-eicosatrienoic acid (eicosatrienoic acid (ETE), 20:3 (n-3)); 8,11,14,17-eicosatetraenoic acid (eicosatetraenoic acid (ETA), 20:4 (n-3)); 5,8,11,14,17-eicosapentaenoic acid (eicosapentaenoic acid (EPA), (20:5 (n-3)); 7,10,13,16,19-docosapentaenoic acid (docosapentaenoic acid (DPA), 22:5 (n-3)); 4,7,10,13,16,19-docosahexaenoic acid (docosahexaenoic acid (DHA), 22:6 (n-3)); 9,12,15,18,21-tetracosapentaenoic acid (tetracosapentaenoic acid, 24:5 (n-3)); and 6,9,12,15,18,21-tetracosahexaenoic acid (tetracosahexaenoic acid, 24:6 (n-3)).

Eicosapentaenoic acid (EPA) and docosahexaenoic acid (DHA) are found in nature in fish oils, and have been used in a variety of dietary/therapeutic compositions. EPA and DHA are preferred omega-3 polyunsaturated fatty acids in the present invention.

Component (1) can include other polyunsaturated fatty acids or derivatives thereof besides omega-3 polyunsaturated fatty acids or derivatives thereof, such as omega-6 or omega-9 polyunsaturated fatty acids or derivatives thereof.

By way of example of polyunsaturated fatty acids and derivatives thereof, EPA, DHA and derivatives thereof are described below. This description also applies to other omega-3 polyunsaturated fatty acids and derivatives thereof.

EPA and DHA Polyunsaturated Fatty Acids

EPA and DHA are omega-3 polyunsaturated fatty acids. As used herein, the term “omega-3 polyunsaturated fatty acid(s)” refers to the fact that EPA and DHA have a carbon-carbon bond in the n-3 position (i.e., the third bond from the methyl end of the molecule). EPA and DHA can be found in nature, and these natural omega-3 polyunsaturated fatty acids frequently have all of their carbon-carbon double bonds in the cis-configuration.

EPA is 5,8,11,14,17-eicosapentaenoic acid (eicosapentaenoic acid or “EPA,” (20:5 (n-3)) and DHA is 4,7,10,13,16,19-docosahexaenoic acid (docosahexaenoic acid or “DHA,” 22:6 (n-3)).

Eicosapentaenoic acid (EPA) and docosahexaenoic acid (DHA) are found in nature in fish oils, and have been used in a variety of dietary/therapeutic compositions.

EPA and DHA Derivatives

As used herein, the terms “EPA derivative(s)” and “DHA derivative(s)” refer to EPA and DHA that have been reacted with another compound or otherwise modified so that the EPA and DHA no longer contains a free carboxylic acid. Examples of EPA and DHA derivatives include salts, esters (such as alkyl esters including, but not limited to, methyl and ethyl esters) and glycerides of omega-3 polyunsaturated fatty acids. The EPA and DHA can also be one or more of the fatty acid moieties in a phospholipid molecule. Since the derivatives are intended to be administered to a subject, they should be pharmaceutically acceptable. As used herein, the term “pharmaceutically acceptable” means that the material to which it refers is not harmful to the subject.

As used herein, the term “glyceride” means a glycerol molecule (i.e., OHCH₂CHOHCH₂OH) in which one, two or all three of the hydroxyls have been esterified with a carboxylic acid, e.g., an omega-3 polyunsaturated fatty acid. Thus, “triglyceride” refers to glycerides in which all three hydroxyls on the glycerol have been esterified with (the same or different) carboxylic acids. “Diglyceride” refers to glycerides in which only two of the hydroxyls on the glycerol have been esterified with (the same or different) carboxylic acids. “Monoglyceride” refers to glycerides in which only one hydroxyl on the glycerol has been esterified with a carboxylic acid.

Omega-3 fatty acids are found in nature in the triglyceride form (a glycerol with three fatty acids attached). The natural triglyceride form as found in raw fish oil cannot be readily separated as it occurs into purified EPA/DHA mixtures by ordinary means such as distillation or crystallization, because the fatty acids are non-uniformly distributed among the triglyceride molecules. There are very few, if any, single triglyceride molecules which are composed of either three EPAs or three DHAs. Typically, there is a DHA, an EPA, and another fatty acid in a triglyceride molecule. So in order to purify fatty acids to increase the proportion of EPA, DHA, or the total fraction of omega-3's, it is necessary to hydrolyze the triglycerides to remove at least some fatty acids from the glycerol.

The triglycerides may be converted by any method known to one skilled in the art without limitation. For example, the triglycerides may be converted by lipase-catalyzed esterification or lipase catalyzed acidolysis with ethyl or lauryl alcohol, which can selectively leave the highest amount of EPA and DHA bonded to glycerols and remove other components, leaving EPA and/or DHA as mono- or di-glycerides. The mono- and di-glycerides can then be separated into fractions with different EPA/DHA ratios, by methods familiar to those skilled in the art such as multiple stage vacuum distillation and/or fractional crystallization in urea. Advantageously, the purified EPA and DHA esters, after concentration, can be reattached to glycerol molecules using enzymatic reacylation to recreate glycerides which are otherwise identical to the original natural triglycerides, except that they are more concentrated in EPA and DHA combined, and they may also have a different ratio of EPA:DHA than the original fish oil. In some embodiments, at least 60% of the omega-3 fatty acids, and preferably 70% or more are converted to the triglyceride form in the reacylation process. The process may be successively repeated with addition of additional catalyst and/or enzyme and additional EPA and DHA until the desired specification proportions are met. About 60% of triglycerides can be made in the first pass of reacylation, with most of the remainder of the product being mono- and di-glycerides.

Polyunsaturated fatty acid triglycerides can be prepared using the following method.

1. Removal of Free Fatty Acids

Raw fish oil in the natural triglyceride molecular form preferably from anchovies and sardines which contain about 18% EPA and 12% DHA is heated to 60° C. to decrease viscosity. Sodium oxide is added to bind with free fatty acids in the oil. The mixture is moved to a separator where sodium oxide bound to free fatty acids (soap) floats to the top and is removed.

The oil is then moved to a second separator where warm water is preferably added to help remove traces of sodium oxide, as sodium oxide partitions to water, yet does not interact with the fish oil.

Citric acid may then be added to support splitting the oil from the combination of water and sodium oxide. The oil is then cooled to 30° C. to protect it from oxidation.

2. Stripping and Purification

Oil is moved to a separate stripping tank, and heated to 200° C. Ethyl esters can be added to support the removal of impurities, which bind to ethyl esters. Impurities such as dioxins, heavy metals, pcbs, fire retardants, furans and others evaporate and are drawn to the middle of the tank where a refrigerating element cools them down and drain them. The added esters are also removed with the impurities.

3. Esterification

The oil is moved to an esterification tank. Ethanol and sodium metal are added. Sodium metal is a catalyst for breaking off fatty acid strands from the glycerol backbone of the triglyceride fatty acid molecule, the free fatty acids then combined with ethanol to form ethyl esters. Water can be added to bind to sodium metal, where the combination of water and sodium metal can be removed.

4. Molecular Distillation

The oil is then moved to a distiller where it is heated to about 120° C. under vacuum. Mono esters and shorter carbon chain molecules move to the middle where they are cooled and drained, leaving longer carbon chains remaining as a concentrate. The process typically increases the key fatty acids by 100% during the first distillation; typically between 30-50% during the second distillation. The process can be repeated, although preferably the process is ideally only repeated once, as when oils undergo heat it can produce oxidation and degradation of the fatty acids in general. Oil waste is also increasing with repeated distillation, making the process less economical.

Oils having higher EPA content can be produced by repeating the molecular distillation step to separate EPA from other fatty acids, including separation from DHA.

5. Reesterification (Reacylation)

The oil is then moved to a reesterification tank where the ethyl ester molecules are reconverted to the triglyceride form, which is the natural form of that fatty acid molecule. 98% of fats ingested by humans are in this natural triglyceride form.

The esterification process takes place under low vacuum at about 80° C. Glycerol is added to form the backbone of the glyceride molecules. Nitrogen can be added from the bottom of the tank to cause oil movement. Lipase enzymes are added as catalysts to facilitate the fatty acids binding to glycerol. The vacuum in the distillation tank removes the ethanol which was previously bound to the fatty acids. The enzymes used are lipases produced from bacteria or yeast. Perhaps the most effective enzymes are Candidan Antarctica lipase, and Chromobacterium Viscosum Lipase; other enzymes that can be used effectively are Psuedomonas, Mucor miehei, and Candida Cylindracea as well as other enzymes may also be used.

The reesterification process typically takes 24 hours, at which point the triglycerides typically reaches 60-65%, the remaining glycerides being diglycerides and monoglycerides. Around 3% of the fish oil will remain as ethyl esters, which can be removed together with the ethanol. Adding additional enzymes and/or continuing the enzymatic process can produce triglyceride molecule concentration of up to 99%. The 60-65% level is probably optimum from an economic point of view.

6. Winterization

The oil in triglyceride form is then moved to a cooling tank at 0° C., where saturated fats, in particular stearic acid are crystallized. The pulp is then pumped to a filter press, where the crystals are removed, essentially removing the vast majority of saturated fats from the oil. Depending on the amount of saturated fats in the oil, approximately 5-10% of the oil is lost during this process.

7. Bleaching

The oil is then removed to a bleaching tank at 60° C., where bleaching earth or bentonite earth is added to the oil. Any water in the oil evaporates due to the temperature. Any remaining impurities (trace minerals, etc) in the oil attach to the bentonite earth. The oil is then run through a bentonite earth filter to remove the bentonite earth together with the impurities.

8. Deodorization

Although not a necessary step, it is advantageous to move the oil to a deodorization tank. The tank contains low vacuum at 120° C. Steam is added at the bottom of the tank, which connects to color and odor molecules (oxidated matter, peroxides) which again travel into the vacuum system and into a residue container. This process gives the oil a neutral color with virtually zero taste and odor.

9. Mixing.

The oil is then moved to a separate storage tank. Depending on the concentration of EPA and DHA desired, various batches can be mixed to yield the concentration desired for the final product.

10. Addition of Antioxidant

Antioxidants, in particular rosemary and mixed tocopherols can be added to the final oil to dramatically reduce the oxidation process.

11. Drumming

The oil is then drummed in stainless steel drums for storage and topped off with nitrogen to remove oxygen and minimize the potential for oxidation.

Sources of the polyunsaturated fatty acids or derivatives thereof (including omega-3 polyunsaturated fatty acids or derivatives thereof) include natural sources including, but not limited to, fish oil (e.g., cod liver oil), flax seed oil, marine oils, sea oils, krill oil, algae and the like. Fish oil is a preferred source.

It is preferred to use a high quality source of omega-3 polyunsaturated fatty acids or derivatives thereof which is rich in omega-3 oils, preferably containing at least 70% omega-3 oils. The oil can also be rich in EPA and may contain some DHA. Preferably, at least 75% of the omega oils are EPA+DHA, and more preferably 85% or more are EPA+DHA, with the majority (if not all) being EPA.

The Red Yeast Rice extract is water soluble and is not soluble in the omega-3 oil. In some embodiments, a dispersing agent is used to keep the Red Yeast Rice extract in suspension. In some embodiments, the dispersing agent is about 70% silica bamboo with lysine made from sunflower oil. A suitable method for making a therapeutic composition of the present invention is to vigorously mix Red Yeast Rice extract with fish oil (containing a suitable amount of component (1)), bamboo (2%), and lysine (3%). The resulting mixture may then be diluted to the desired omega-3 oil to Red Yeast Rice extract ratio. In some embodiments, the omega-3/Red Yeast Rice extract is then encapsulated in soft gelatin capsules for dispensing. The capsules are typically of such a size that an integral number of capsules comprise a daily dosage of the mixture.

In some embodiments, a dosage of the therapeutic composition of the present invention further includes antioxidants such as rosemary, vitamin E, astaxanthine, carnitine, ascorbyl palmitate, tocopherols or other antioxidants known in the art for stabilizing fish oil and/or omega-3 polyunsaturated fatty acids or derivatives thereof.

Comparison between Red Yeast Rice Extract and Lovastatin

Red Yeast Rice extract contains monacolin K, the lactone form of the statin drug Mevacor® (lovastatin). Red Yeast Rice extract has been tested in clinical trials at daily dosages of 1.2 g and 2.4 g. The monacolin K content of the Red Yeast Rice extract used in the clinical trials was 0.20% of the Red Yeast Rice extract. The monacolin K dose was therefore 2.4 to 4.8 mg/day. At 2.4 mg/day of monacolin K, the total cholesterol, LDL cholesterol, and triglycerides dropped by 23%, 31%, and 34% respectively. At 4.8 mg/day, the reduction was 17%, 23%, and 16% respectively (see Monograph by Thorne Research Inc., Alternative Medicine Review, 2004, 9:1).

Lovastatin has been shown to have a cholesterol lowering effect in doses ranging from 5 to 80 mg/day (see Bates et al., “Effectiveness of low dosage lovastatin in lowering serum cholesterol. Experience with 56 patients.” Archives of Internal Medicine 1990, 150:1947-1950). A study was performed to show the effectiveness of low-dose lovastatin in lowering serum cholesterol (see Heber et al., “Cholesterol lowering effects of proprietary Chinese red yeast rice dietary supplement,” Ann J Clin Vutr 1999, 69:23 1 -236). Fifty-six patients were given 20 mg/day of lovastatin for 24 weeks. Total cholesterol fell by 26% and triglycerides fell by 12%.

Mevacor® (lovastatin) in its package insert reported extensive clinical trials at dosages of 10, 20, and 40 mg/day. Total cholesterol was reduced in the range from 16-24%, LDL was reduced by 21-32%, and triglycerides were reduced by 10 to 6% (higher reduction observed at lower dosage).

It should be noted that Red Yeast Rice extract at a dosage of 2.4 mg/day of monacolin K produced better lipid reducing results than Mevacor at 10-40 mg/day. It is, therefore, unlikely that the lipid lowering effects with Red Yeast Rice result from the monacolin K content alone of Red Yeast Rice, but are probably attributable in whole or in part to the other monacolins, sterols (beta-sitosterol, campesterol, sigmasterol, and sapogenin), isoflavones, and monounsaturated fatty acids Red Yeast Rice extract (see Durington et al., “An omega-3 polyunsaturated fatty acid concentration administered for one year decreased triglycerides in simvastin treated patients with CM,” Heart 2001, 85(5) 544-548) This is a particular advantage since the lower dosage of Red Yeast Rice extract containing HMG-CoA reductase inhibitor contributes to reduced side effects as well.

Red Yeast Rice extract can be purchased as a nutritional supplement in the United States. Preferred sources include DRACO Natural Products (539 Parrott St., San Jose, Calif. 95112) Red Yeast Rice Extract 10:1, and the Thorne Research product, Choleast. Purchased Red Yeast Rice preferably should contain about 0.2 wt. % or more of monacolin K and about 0.5 wt. % or more of total monacolins.

The compositions of this invention can contain other ingredients besides the ingredients recited above. These include, but are not limited to, flavor agents, fillers, surfactants (e.g., polysorbate 80 and sodium lauryl sulfate), color agents including, e.g., dyes and pigments, sweeteners, antioxidants and additional ingredients.

Flavor Agents

Useful flavor agents include natural and synthetic flavoring sources including, but not limited to, volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, banana, grape, berry, strawberry, raspberry, blueberry, blackberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof, chocolate, cocoa, almond, cashew, macadamia nut, coconut, mint, chili pepper, pepper, cinnamon, vanilla, tooty fruity, mango and green tea. Mixtures of two or more flavor agents may also be employed. When a flavor agent is used, the amount employed will depend upon the particular flavor agent used. However, in general, the flavor agent can constitute from about 5% to about 50% by weight of the composition.

Color Agents

Useful color agents include, e.g., food, drug and cosmetic (FD&C) colors including, e.g., dyes, lakes, and certain natural and derived colorants. Useful lakes include dyes absorbed on aluminum hydroxide and other suitable carriers. Mixtures of color agents may also be employed. When a color agent is employed, the amount used will depend upon the particular color agent used. However, in general, the color agent can constitute from about 0.5% to about 5% by weight of the composition.

Sweetening Agent

Natural and/or artificial sweetening agents can also be added to the composition. Examples of sweeteners include sugars such as sucrose, glucose, invert sugar, fructose, and mixtures thereof, saccharin and its various salts (e.g., sodium and calcium salt of saccharin), cyclamic acid and its various salts, dipeptide sweeteners (e.g., aspartame), dihydrochalcone, and sugar alcohols including, e.g., sorbitol, sorbitol syrup, mannitol and xylitol, and combinations thereof Natural sweeteners that can be employed include, but are not limited to, luo han, stevia or mixtures thereof. Luo han sweetener is derived from luo han guo fruit (siraitia grosvenorii) that is mainly found in China. It is about 300 times sweeter by weight than sucrose. Luo han is commercially available from, e.g., Barrington Nutritionals (Harrison, N.Y.). Stevia is derived from a South American herb, Stevia rebaudiana. It can be up to about 300 times sweeter than sucrose. Because luo han and stevia have such a sweet taste, only a small amount need be used in the composition. When a sweetening agent is employed the amount used will depend upon the particular sweetening agent used. However, in general, the sweetening agent can constitute from about 0.0005% to about 30%, by weight of the composition. When a sweetener having a very sweet taste, such as luo han or stevia, is used, small amounts such as about 0.0005% to about 0.1% (for example about 0.005% to about 0.015% or about 0.002% to about 0.003%) by weight can be used.

Additional Ingredients

The compositions of the present invention can contain additional ingredients. Examples of such additional ingredients include, but are not limited to, vitamins, minerals and/or herbs.

As used herein, the term “vitamin” refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ10), pyridoxal phosphate, biocytin, tetrahydrofolic acid, coenzyme B12, lipoyllysine, 11-cis-retinal, and 1,25-dihydroxycholecalciferol. The term vitamin(s) also includes choline, camitine, and alpha, beta, and gamma carotenes.

As used herein, the term “mineral” refers to inorganic substances, metals, and the like required in the human diet. Thus, the term “mineral” as used herein includes, without limitation, calcium, iron, zinc, selenium, copper, iodine, magnesium, phosphorus, chromium and the like, and mixtures thereof. Compounds containing these elements are also included in the term “mineral.”

As used herein, the term “herb” refers to organic substances defined as any of various often aromatic plants used especially in medicine or as seasoning. Thus, the term “herb” as used herein includes, but is not limited to, black currant, ginsing, ginko bilboa, cinnamon, and the like, and mixtures thereof.

Other ingredients that can be used include antioxidants, glucosamine and mixtures thereof.

The compositions of this invention are suitable for therapeutic and/or nutritional purposes in treating a subject in need of such treatment. As used herein, the term “subject” includes, but is not limited to, a non-human animal, such as a cow, monkey, horse, sheep, pig, chicken, turkey, quail, cat, dog, mouse, rat, rabbit, or guinea pig; and a human.

The amount of the composition of the invention that is effective will vary depending upon the condition being treated, and can be determined by standard clinical techniques. In addition, in vitro or in vivo assays can optionally be employed to help identify optimal dosage ranges. The precise dose to be employed will also depend on the relative amounts of the components of the compositions of the invention, route of administration, and the seriousness of the condition being treated and should be decided according to the judgment of the practitioner and each subject's circumstances. However, suitable effective dosage amounts for the compositions of the invention typically are at least about 2 grams per day, typically administered in the form of capsules containing at least about 1 gram of the composition per capsule.

The compositions of the present invention comprise component (1) and component (2) wherein components (1) and (2) are present in amounts effective to help reduce LDL cholesterol and/or triglycerides in a subject. The phrase “present in amounts effective to help reduce LDL cholesterol and/or triglycerides in a subject” as used herein means that components (1) and (2) are used in an amount, individually and in combination, effective for a therapeutic, preventive or nutritional activity in a subject that promotes or supports reduction of LDL cholesterol and/or triglycerides in the subject. By “promote or support reduction of LDL cholesterol and/or triglycerides in a subject” is meant the compositions help lower (or at least help maintain) LDL cholesterol and/or triglyceride levels in the subject. By “amount individually and in combination effective” is meant that each individual component is present in an amount sufficient to perform its function as well as the overall composition being in an amount sufficient to perform its overall function.

The form in which the composition of the invention is administered to the subject is not critical. Typically, the composition is administered as a liquid, as a dispersion or in a capsule. Typically, the composition is administered in the form of individual doses. As used herein, the term “dose” includes both the case where component (1) and component (2) are administered together (such as in the form of a capsule containing both components), and the case where component (1) and component (2) are administered separately (but, typically, at essentially the same time). It is preferred that components (1) and (2) be administered together, such as in the form of a single capsule.

In some embodiments, the composition of the invention is administered in the form of a daily dose. However, depending on the severity of the condition being treated, this may not be required, and the period between administration of the doses may be longer than one day. In addition, the term “administer” includes both the case where a third party administers the dose to the subject and the case where the subject self-administers the dose.

The following tables show formulations in accordance with the present invention (active ingredients only per 1000 mg capsule) in accordance with the present invention:

TABLE 1

| COMPONENT FORMULATION | FORMULA I | FORMULA II |
|---|---|---|
| Fish Oil (mg) | 800 | 900 |
| Red Yeast Rice (RYR) (mg) | 200 | 100 |
| Total | 1000 | 1000 |
| EPA* (mg) | 600.0 | 810.0 |
| DHA* (mg) | 80.0 | 45.0 |
| Monacolin K (mg) | 1.6 | 2.4 |
| Red Yeast Rice | | |
| Monacolin K (wt. % of RYR) | 0.8% | 2.4% |

*Triglyceride form

TABLE 2

| COMPONENT FORMULATION | FORMULA III |
|---|---|
| Fish Oil (mg) | 600 |
| Red Yeast Rice (RYR) (mg) | 400 |
| Total | 1000 |
| EPA* (mg) | 390.0 |
| DHA* (mg) | 30.0 |
| EPA:DHA (wt. ratio) | 13.1 |
| Monacolin K (mg) | 3.2 |
| Red Yeast Rice | |
| Monacolin K (wt. % of RYR) | 0.8% |

*Triglyceride form

TABLE 3

| COMPONENT FORMULATION | FORMULA IV | FORMULA V |
|---|---|---|
| Fish Oil (mg) | 700 | 800 |
| Red Yeast Rice (RYR) (mg) | 300 | 200 |
| Total | 1000 | 1000 |
| EPA* (mg) | 560.0 | 720.0 |
| DHA* (mg) | 35.0 | 40.0 |
| EPA:DHA (wt. ratio) | 16:1 | 18:1 |
| Monacolin K (mg) | 3.6 | 4.0 |
| Red Yeast Rice | | |
| Monacolin K (wt. % of RYR) | 1.2% | 2.0% |

*Triglyceride form

In some embodiments, a daily dose of the therapeutic compositions of the present invention comprises four 1000 mg doses of Formula I, II, III, IV, or V in the tables above. In some embodiments, the daily dose comprises four capsules, each containing 1000 mg of Formula I, II, III, IV, or V. In some embodiments, a daily dose is taken, the dose comprising 3200 mg of fish oil containing component (1) and sufficient Red Yeast Rice extract to provide 6.4 mg of monacolin K Such a dose could include 2400 mg EPA (triglyceride form), 320 mg DHA (triglyceride form) and 6.4 mg monacolin K. Here, the weight ratio of fish oil to Red Yeast Rice extract is 4:1.

In some embodiments, a daily dose is taken, the dose comprising 3600 mg of fish oil containing component (1) and sufficient Red Yeast Rice extract to provide 9.6 mg of monacolin K Such a dose could include 3240 mg EPA (triglyceride form), 180 mg DHA (triglyceride form) and 9.6 mg monacolin K. Here, the weight ratio of fish oil to Red Yeast Rice extract is 9:1.

Although the present invention has been described in considerable detail with reference to certain versions thereof, other versions are possible. Therefore the spirit and scope of the appended claims should not be limited to the versions presented herein.

1. A therapeutic composition comprising a composition (1) comprising at least one polyunsaturated fatty acid, at least one pharmaceutically acceptable derivative of a polyunsaturated fatty acid or mixtures thereof and a composition (2) comprising Red Yeast Rice extract comprising about 0.8 wt. % or more monacolin K.

2. The therapeutic composition of claim 1 wherein component (1) comprises at least one omega-3 polyunsaturated fatty acid, or at least one pharmaceutically acceptable omega-3 polyunsaturated fatty acid derivative or mixtures thereof

3. The therapeutic composition of claim 2 wherein component (1) comprises pharmaceutically acceptable derivatives of EPA and pharmaceutically acceptable derivatives of DHA.

4. The therapeutic composition of claim 3 wherein pharmaceutically acceptable derivatives of EPA and pharmaceutically acceptable derivatives of DHA are present in a weight ratio of about 3:1; about 3.2:1; about 3.3:1; about 5:1; or about 5.5:1.

5. The therapeutic composition of claim 3 wherein the derivatives of EPA and derivatives of DHA are selected from the group consisting of esters, alkyl esters, glycerides and phospholipids.

6. The therapeutic composition of claim 5 wherein the derivatives of EPA and derivatives of DHA are monoglycerides, diglycerides, triglycerides or mixtures thereof.

7. The therapeutic composition of claim 6 wherein component (1) is a mixture comprising about 35 wt. % triglycerides of EPA and about 25 wt. % triglycerides of DHA.

8. The therapeutic composition of claim 6 wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the EPA and DHA and the balance is at least about 80% mono- and di-glycerides.

9. The therapeutic composition of claim 1 wherein component (2) comprises about 0.8 wt. % to about 25 wt. % monacolin K.

10. The therapeutic composition of claim 1 wherein the weight ratio of component (1) to component (2) is in the range between about 1:1 to about 10:1; or in the range between about 4:1 to about 10:1.

11. The therapeutic composition of claim 1 further comprising a dispersing agent.

12. The therapeutic composition of claim 11 wherein the dispersing agent comprises lysine and bamboo.

13. The therapeutic composition of claim 1 further comprising an antioxidant.

14. The therapeutic composition of claim 13 wherein the antioxidant is chosen from the group consisting of rosemary, vitamin E, astaxanthine, carnitine, ascorbyl palmitate, and tocopherols.

15. The therapeutic composition of claim 1 wherein a daily dose of the therapeutic composition comprises:
(i) about 2200 mg of component (1) and a sufficient amount of component (2) to provide at least about 6.0 mg of monacolin K; or
(ii) about 2700 mg of component (1) and a sufficient amount of component (2) to provide about 6.4 mg of monacolin K; or
(iii) component (1) comprising about 2400 mg of a pharmaceutically acceptable derivative of EPA and about 320 mg a pharmaceutically acceptable derivative of DHA; and a sufficient amount of component (2) to provide about 6.4 mg of monacolin K; or
(iv) component (1) comprising about 3240 mg of a pharmaceutically acceptable derivative of EPA and about 180 mg a pharmaceutically acceptable derivative of DHA; and a sufficient amount of component (2) to provide about 9.6 mg of monacolin K.

16. A method of reducing serum cholesterol, triglycerides or both in a subject comprising administering to the subject an effective amount of a dosage comprising a composition (1) comprising at least one polyunsaturated fatty acid, or at least one pharmaceutically acceptable derivative of a polyunsaturated fatty acid or mixtures thereof; and a composition (2) comprising Red Yeast Rice extract comprising about 0.8 wt. % or more monacolin K.

17. The method of claim 16 wherein component (1) comprises at least one omega-3 polyunsaturated fatty acid, or at least one pharmaceutically acceptable omega-3 polyunsaturated fatty acid derivative or mixtures thereof.

18. The method of claim 17 wherein component (1) comprises pharmaceutically acceptable derivatives of EPA and pharmaceutically acceptable derivatives of DHA.

19. The method of claim 18 wherein pharmaceutically acceptable derivatives of EPA and pharmaceutically acceptable derivatives of DHA are present in a weight ratio of about 3:1, about 3.2:1, about 3.3:1, about 5:1 or about 5.5:1.

20. The method of claim 18 wherein the derivatives of EPA and derivatives of DHA are selected from the group consisting of esters, alkyl esters, glycerides and phospholipids.

21. The method of claim 20 wherein the derivatives of EPA and derivatives of DHA are monoglycerides, diglycerides, triglycerides or mixtures thereof.

22. The method of claim 21 wherein component (1) is a mixture comprising about 35 wt. % triglycerides of EPA and about 25 wt. % triglycerides of DHA.

23. The method of claim 21 wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the EPA and DHA and the balance is at least about 80% mono- and di-glycerides.

24. The method of claim 16 wherein component (2) comprises about 0.8 wt. % to about 25 wt. % monacolin K.

25. The method of claim 16 wherein the weight ratio of component (1) to component (2) is in the range between about 1:1 to about 10:1; or in the range between about 4:1 to about 10:1.

26. The method of claim 16 further comprising a dispersing agent.

27. The method of claim 26 wherein the dispersing agent comprises lysine and bamboo.

28. The method of claim 16 further comprising an antioxidant.

29. The method of claim 28 wherein the antioxidant is chosen from the group consisting of rosemary, vitamin E, astaxanthine, carnitine, ascorbyl palmitate, and tocopherols.

30. The method of claim 16 wherein a daily dose of the therapeutic composition comprises:
(i) about 2200 mg of component (1) and a sufficient amount of component (2) to provide at least about 6.0 mg of monacolin K, or
(ii) about 2700 mg of component (1) and a sufficient amount of component (2) to provide about 6.4 mg of monacolin K, or
(iii) component (1) comprising about 2400 mg of a pharmaceutically acceptable derivative of EPA and about 320 mg a pharmaceutically acceptable derivative of DHA; and a sufficient amount of component (2) to provide about 6.4 mg of monacolin K, or
(iv) component (1) comprising about 3240 mg of a pharmaceutically acceptable derivative of EPA and about 180 mg a pharmaceutically acceptable derivative of DHA; and a sufficient amount of component (2) to provide about 9.6 mg of monacolin K.
